(19) United States
(12) Patent Application Publication
(10) Pub. No.: US 2007/0190547 A1
(43) Pub. Date: Aug. 16, 2007

(54) RESULTS FOR QUALITY CHARACTERISTIC VALUES FOR NUCLEIC ACID PREPARATIONS

(76) Inventor: David E. Kohne, San Diego, CA (US)

Correspondence Address: WESLEY B. AMES, 7031 LOS VIENTOS SERENOS, ESCONDIDO, CA, 92029, US

(21) Appl. No.: 11/619,195

(22) Filed: Jan. 3, 2007

Related U.S. Application Data
(60) Provisional application No. 60/755,170, filed on Dec. 30, 2005.

(51) Int. Cl. C12Q 1/68; G06F 19/00

(52) U.S. Cl. 435/6; 702/20

(57) ABSTRACT

The invention provides a method for producing improved quality characteristic results for purified nucleic acid preps of virtually any kind. Such results include the quantitation, purity, integrity, and functional homogeneity, quality characteristic results. Further, the invention provides a method for producing improved application results for applications which utilize nucleic acid preps. Such results are improved in accuracy, reproducibility, intercomparability, interpretability, and utility, relative to prior art produced results.

### RELATED APPLICATIONS

This application claims the benefit of Kohne, U.S. Provisional Appl. 60/755,710, filed Dec. 30, 2005, which is incorporated herein by reference in its entirety.

### FIELD OF THE INVENTION

The present invention relates to improvements in quality characteristic values for nucleic acid preparations.

The invention relates to the determination of characteristics which describe the quality of a nucleic acid preparation of any kind. These characteristics are herein termed nucleic acid preparation quality characteristics. Such nucleic acid preparations include chemically or enzymatically synthesized nucleic acid preparations of all kinds, as well as natural nucleic acid preparations of all kinds, including those from virus, and prokaryote and eukaryote cells of all kinds. The nucleic acid preparation quality characteristics include the following. (i) The amount of nucleic acid present in the nucleic acid preparation, i.e. the nucleic acid preparation quantitation quality characteristic. (ii) The purity of the nucleic acid preparation, i.e. the nucleic acid preparation purity quality characteristic. (iii) The integrity of the nucleic acid molecules comprising the nucleic acid preparation, i.e. the nucleic acid preparation integrity quality characteristic. (iv) The functional homogeneity of the nucleic acid molecules present in the nucleic acid preparation, i.e. the nucleic acid preparation functional homogeneity quality characteristic. The invention further relates to any application which utilizes a nucleic acid preparation, or utilizes the results obtained with a nucleic acid prep.

### BACKGROUND OF THE INVENTION

The following discussion is provided solely to assist the understanding of the reader, and does not constitute an admission that any of the information discussed or references cited constitute prior art to the present invention.

To simplify this discussion, a nucleic acid preparation refers to a preparation of natural or chemically or enzymatically synthesized RNA or DNA or modified RNA or modified DNA, or other nucleic acid.

Nucleic acid preparation characteristics which can be used to define the quality of a nucleic acid preparation include the following. (a)The concentration or amount of nucleic acid present in the preparation. Here this is termed the nucleic acid prep quantitation quality characteristic. (b) The purity of the preparation with regard to the fraction of the preparation which consist of the nucleic acid of interest. This is termed the nucleic acid prep purity quality characteristic. (c) The integrity of the nucleic acid molecules which are present in the preparation. Here, the integrity of a nucleic acid preparation indicates whether the nucleic acid molecules which make up the preparation have the desired or intended degree molecular of intactness. This is termed the nucleic acid prep integrity quality characteristic. (d) The functional homogeneity of the nucleic acid molecules which comprise the nucleic acid prep. The functional homogeneity is a measure of the fraction of nucleic acid molecules present in the prep will do what they are designed to do. Almost always the nucleic acid molecules are designed to specifically hybridize to one or more intended target nucleic acid molecules. This is termed the nucleic acid prep functional homogeneity quality characteristic. Each of these nucleic acid preparation characteristics can be measured in semi-quantitative or quantitative terms. Prior art commonly attempts to measure accurate quantitative values for the quantitation and purity and integrity nucleic acid prep quality characteristics (1-16). Prior art only rarely attempts to measure accurate quantitative values for functional homogeneity quality characteristic.

Prior art routinely attempts to determine accurate values for the quantitation quality characteristic of nucleic acid preps. A variety of methods are used for this purpose. By far the most widely used methods involve determining the amount of nucleic acid present in a prep with spectrophotometric or fluorescent methods (1-4).

The spectrophotometric method for quantitation relies on the ability of nucleic acids to absorb ultraviolet (UV) light wavelengths up to about 305 nanometers (nm). Nucleic acids do not absorb light which has a wave-length of greater than about 305 nm. The absorption maxima for nucleic acids occur at roughly 260 nm. The absorption maximum varies somewhat for nucleic acids of different gene composition. The half maximum absorption of nucleic acids occurs at about 280 nm. The almost universally used prior art method of spectrophotometric determination of the quantitation characteristic of a nucleic acid prep involves measuring the absorption of a dilution of the nucleic acid prep in a solution, usually water, and determining the absorbance of the solution at 260 nm. The OD260 value represents the absorbance of a 1 cm path-length of solution. This OD260 absorbance values is generally termed an optical density at 260 nm value, or an OD260 value, and this terminology will be used herein. The nucleic acid OD260 value for the sample is then converted to micrograms (mcg) of nucleic acid per milliliter (ml) by using well known conversion factors (1-4). It is well known that the quantitative values for these conversion factors can vary significantly for single and double strand nucleic acids and nucleic acids of different composition, as well as for the same nucleic acid in solutions of different pH and composition (1-4,10). There is at present no generally accepted solution of known composition and pH which is used to determine the quantitation characteristics of nucleic acid preps.

A spectrophotometric OD260 determination is virtually always done on a purified nucleic acid prep. Depending on the cell or other sample processed, and the purification process used, the purity of the resulting nucleic acid prep can vary greatly. Varying amounts of protein or high molecular weight polysaccharides may be present in the purified nucleic acid prep (1-4, 17). Other contaminants often include salts, reagents, and particulate material, associated with the isolation procedure and devices (1-13). The presence of significant amounts of protein, high molecular weight polysaccharide or other particulate substances, and non-nucleic acid low molecular weight UV absorbing substances, can cause OD260 measured nucleic acid concentration to be significantly inaccurate. The presence of such contaminants in purified nucleic acid preps is not uncommon. It is generally believed that the most common contaminant of purified nucleic acid preps is one or more proteins of an unspecified type. The presence of large amounts of protein in a purified nucleic prep has little effect on the accuracy of the OD260 determined nucleic acid concentration value for the nucleic acid solution (7). The presence in a purified in a purified nucleic acid preparation of about 80% or 50% protein by weight, causes the measured OD260 derived nucleic acid concentration to deviate from accuracy by 1.1 fold and 1.04 fold respectively. This occurs because the OD260 value for separate 1 milligram per ml solutions of pure protein and pure nucleic acid equals about 0.5 and 20 respectively. Prior art information concerning the actual amounts of protein present in different purified nucleic acid preps is very limited. The presence of UV absorbing low molecular weight substances is common in purified nucleic acid preps. Examples of such substances are phenol and thiocyanates. A variety of methods for minimizing or eliminating the effect of such low molecular weight contaminants are recommended by the prior art. The presence of high molecular weight particulate substance contaminants in purified nucleic acid preps is also common. Prior art is aware of the existence of such contaminants and is aware that such contaminants scatter light at all wave-lengths, including 260 nm and 280 nm. Further, prior art is aware that the presence in a nucleic acid solution of such light scattering substances can be detected by measuring the OD of the solution at a wavelength where neither proteins nor nucleic acids absorb light. Herein such a light scattering substance is termed and LSS. However, prior art methods which describe a valid method for correcting a purified nucleic acid preparation OD260 value for the presence of LSS have not been discovered.

An alternate nucleic acid quantitation method relies on the increased fluorescent signal obtained from fluorescent dye: nucleic acid complexes, relative to the signal from non-complexed dyes (1,4,11). A variety of different dyes are available for this purpose. This method is significantly more sensitive than the OD260 method. The method is sometimes described as being more accurate than the A260 method. However, there is little information to support this claim. Done properly, the OD260 method should be significantly more accurate and reproducible than the dye methods. This should be especially true in the presence of protein. The prior art determination of nucleic acid prep purity is generally done spectrophotometrically by determining for a purified nucleic acid solution the OD260 and OD280 values, and then determining the OD260/OD280 ratio value for the nucleic acid solution. The OD260/OD280 value is generally believed to be an effective measure of the purity of the purified nucleic acid preparation (1-13). It has been reported that pure preparations of DNA and RNA have OD260/OD280 ratios of 1.8 and 2.0 respectively (1). Purified RNA preparations with OD260/OD280 ratio values of between 1.8 and 2.0 are regarded by some as being acceptably pure(1-16). Purified RNA preparations with OD260/OD280 values of greater than 2 have been reported (1, 10, 12, 17, 18, 19). The OD260/OD280 ratio value is also significantly affected by the composition and pH of the measuring solution (10). There is no generally accepted solution of known composition and pH which is used to determine OD260/OD280 ratios. The presence of contaminating low molecular weight substances such as phenol, thiocyanates, and other salts can also affect the OD260/OD280 value for a nucleic acid prep. A variety of methods for minimizing or eliminating the presence of such low molecular weight compounds in purified nucleic acid preps have been reported. The presence of significant amounts of LSS in a purified nucleic acid prep is known to affect the OD260/OD280 ratio value for the purified nucleic acid prep. Further, prior art is aware that the presence of LSS in a purified nucleic acid preparation can be detected by measuring the solution OD at a wavelength where neither proteins nor nucleic acids absorb light. However, prior art methods for correcting a purified nucleic acid solution measured OD260/OD280 ratio for the presence of LSS, have not been discovered.

A variety of well known methods exist for the determination of the integrity of purified nucleic acid preps. These include centrifugation based density gradient separation methods and a variety of electrophoresis based methods, including gel electrophoresis and capillary gel electrophoresis (1, 20-22). These methods are most effective when used in a mode where the nucleic acids are in a completely denatured state during the integrity analysis. Prior art characterizes the integrity of a nucleic acid preparation in various ways. Characterization of the integrity of purified total cell RNA is often done by determining the ratio amounts of large (28S or 23S), or small (18S or 16S), ribosomal RNA which can be detected in the cell sample total RNA preparations (12). The large/small ratio value of 2 or more has been used to indicate a total RNA prep which is of high quality and integrity and is essentially undegraded. The lower this ratio, the more degraded the total RNA prep is, and the lower the quality and integrity of the RNA prep. It is known that ribosomal RNAs are generally more resistant to degradation than mRNAs. Therefore, this ribosomal RNA ratio measure is best used as a qualitative or semi-quantitative method of determining the quality and integrity of the mRNA molecules present in the total RNA prep. Prior art characterization of the integrity of isolated mRNA preps is often done by determining the mRNA molecule nucleotide length distribution profile for the mRNA prep, and comparing it to the mRNA molecule nucleotide length distribution profile for an isolated cell sample mRNA prep which is known to be undegraded. Prior art generally believes and practices that the average mRNA nucleotide sequence length for a typical undegraded mammalian cell mRNA prep is about 1800 nucleotides. Relative to the undegraded mRNA average nucleotide length, the lower the average mRNA nucleotide length for an mRNA prep, the more degraded the mRNA prep is, and the lower the quality and integrity of the mRNA prep. Because the measured quality or integrity indicator reflects the average mRNA molecules nucleotide length for a complex population of different sized mRNA molecules, this method is essentially a qualitative or semi-quantitative method for quality or integrity determination.

There are a variety of prior art methods for the in vitro production of a particular gene's RNA or mRNA molecules in large quantity from DNA clones which contain the particular gene's DNA (23, 24). Such RNA or mRNA molecules are enzymatically synthesized in vitro and then purified to produce purified particular mRNAs or RNA preps. It is desired or intended that such an RNA prep be composed of a population of RNA molecules all of which have the same nucleotide length and nucleotide sequence. Since such a prep would consist of only RNA molecules of the intended nucleotide length, it can be characterized as having 100% integrity. Because of known imperfections which are associated with the RNA production process, RNA molecules of different nucleotide length are almost always, if not always, present in the purified RNA prep. The degree of RNA molecule nucleotide sequence length heterogeneity can vary greatly, and a significant fraction of the total purified particular gene RNA molecule prep may be composed of RNA molecules which have a non-intended or desired nucleotide length. Prior art characterization of the quality and integrity of such purified particular gene RNA preparations, is often done by determining the RNA molecule nucleotide length distribution profile for the particular gene RNA prep and determining the average RNA molecule nucleotide length and a measure of the distribution of the RNA molecule nucleotide lengths for the RNA prep. Prior art methods for determining the average RNA molecule nucleotide length can be facilitated by using other different RNAs of known nucleotide length as molecular weight markers, as is often done. Methods for determining the average RNA nucleotide length and nucleotide length distribution were discussed earlier. Note that because the intended particular gene RNA molecule population represents only one nucleotide sequence, the interpretation of the measured average RNA molecule nucleotide length distribution profile is much simpler than for isolated cell mRNA preps. Because of this, the following characteristics are associated with particular gene RNA preps which have perfect integrity or 100% integrity. (a) The average RNA molecule nucleotide length is equal to the intended nucleotide length. (b) The RNA molecule nucleotide length distribution profile is identical to the RNA molecule nucleotide length distribution profile expected for a population of the particular gene RNA molecules which consists only of RNA molecules of the intended nucleotide length and nucleotide sequence. Note that for a particular gene RNA prep condition (a) May be met while condition (b) is not. Such an RNA prep has a higher integrity than an RNA prep which meets neither condition. Note that the above described approach for determining the integrity if purified particular gene RNA molecules is also used to characterize the integrity of chemically synthesized RNA and DNA molecules. Prior art rarely, if ever, determines the status of (b) For a particular gene RNA prep.

The prior art approach for determining the integrity of cell genomic DNA preps is very similar to the above described approach for determining the integrity of isolated cell mRNA. The prior art approach for determining the intensity of in vitro chemically or enzymatically synthesized particular gene DNA molecules or particular DNA molecules, is very similar to the above described approach for determining the integrity of in vitro enzymatically or chemically synthesized particular gene RNA molecules.

Prior art rarely addresses any aspect of the functional homogeneity characteristic of cell sample derived RNA or DNA preps, or chemically or enzymatically synthesized nucleic acid preps. Further, when this issue is addressed it is done incompletely and qualitatively.

Generally prior art produces a nucleic acid prep in order to use the prep for a particular application which requires the use of a nucleic acid prep. Such particular applications include gene expression analysis and gene expression comparison applications, genomic and other DNA analysis, production of clones for a wide variety of purposes, and others. Prior art believes and practices that accurate knowledge of one or more of the quality characteristics of a nucleic acid prep is necessary in order to obtain accurate and interpretable results for the particular application which the nucleic acid prep is used for. As an example, for gene expression analysis and gene expression comparison analysis of all kinds, prior art routinely attempts to determine the QQC and PQC values for the analyzed nucleic acid preps, and less frequently attempts to determine the IQC for the analyzed nucleic acid preps. These nucleic acid prep quality characteristics are determined because prior art believes it is necessary to know accurate values for these nucleic acid prep quality characteristics in order to obtain accurate and interpretable results for the particular application which uses the nucleic acid prep.

Often, prior art produced particular application results are used as part of a further particular application, to obtain further particular application results. As an example, gene expression analysis and gene expression comparison analysis results are often used in data mining and systems biology processes to produce data mining and systems biology results. Results of these and other further particular applications are often used for other purposes such as drug discovery, drug validation, drug evaluation, drug manufacturing, drug prescription, toxicology evaluation, genetic evaluation, and others. Clearly, the accuracy and interpretability of the quality characteristics of the nucleic acid preps which underpin these applications is important for the accuracy, interpretability, intercomparability, reproducibility, and utility of these application results.

### SUMMARY OF THE INVENTION

The invention provides methods and means for producing nucleic acid preparation quality characteristic information which is known to be improved relative to prior art produce quality characteristic information. The invention pertinent quality characteristics of a nucleic acid preparation of any kind include the nucleic acid prep quantitation characteristic, the nucleic acid prep purity characteristic, the nucleic acid prep integrity characteristic, and the nucleic acid preparation functional homogeneity characteristic. The invention produced nucleic acid preparation quality characteristic information is, relative to prior art produced nucleic acid preparation quality characteristic information, improved in one or more of accuracy and/or interpretability and/or reproducibility and/or normalization completeness and/or utility.

The invention further provides methods and means for producing improved results for particular applications which utilize nucleic acid preps. The use of one or more invention improved nucleic acid prep quality characteristic values for a particular application which uses a nucleic acid prep to produce results, produces particular application results which are improved in accuracy, reproducibility, intercomparability, interpretability, and utility, relative to prior art produced particular application results.

The invention also provides methods and means for producing improved results for further particular applications which utilize the above described improved particular application results. The use of one or more invention improved particular application results in a further particular application which uses such results, produces further particular application results which are improved in accuracy, reproducibility, intercomparability, interpretability, and utility, relative to prior art produced particular application results.

Thus, in one aspect, the invention concerns a method for producing improved quantitation quality characteristic (QQC) and/or purity quality characteristic (PQC) results for one or more nucleic acid preps, which are improved in one or more of normalization and accuracy and interpretability, relative to prior art produced QQC and PQC results for the one or more nucleic acid preps, where the method involves
(a) obtaining (e.g., producing) a purified nucleic acid prep which does not contain significant amounts of low molecular weight UV absorbing contaminant molecules;
(b) measuring the nucleic acid prep absorbance values at wavelengths characteristic of nucleic acid absorbance and light scattering (e.g., 260 nm and 320 nm) and preferably also at a wavelength characteristic of protein absorbance (e.g., 280 nm) and/or small molecule contaminant absorbance (e.g., 230 nm), in a measuring solution of known composition and pH;
(c) normalizing the nucleic acid preps absorbance values (e.g., OD230, OD260, and/or OD280 values) for the measured absorb wavelength characteristic of light scattering (e.g., 320 nm); and
(d) determining a non-light scattering substance related PQC value for the nucleic acid prep from the normalized OD260/OD230 and OD260/OD280 ratio values, and/or
(e) converting the nucleic acid prep normalized OD260 value to micrograms of nucleic acid per ml or other concentration units using a conversion factor which is accurate for the nucleic acid prep measuring solution, to produce a nucleic acid prep QQC value.

In particular embodiments, either the nucleic acid prep OD230 or OD280 value is not determined, and either the nucleic acid prep OD260/OD280 ratio or the nucleic acid prep OD260/OD280 ratio value is not determined; neither the nucleic acid prep OD230 value nor OD280 value is determined and neither the nucleic acid prep OD260/OD230 ratio value nor OD 260/OD280 ratio value is determined.

In particular further embodiments of the preceding, the method also involves determining either or both of the nucleic acid prep integrity quality characteristic (IQC) value, and functional homogeneity quality characteristic (FHQC) value for the nucleic acid prep.

Likewise, in further embodiments of the preceding,
a) The nucleic acid prep is a cell total RNA or isolated mRNA or cell genomic DNA nucleic acid prep, or
b) The nucleic acid prep is a cDNA or cRNA or a RNA or amplified genomic or other DNA nucleic acid prep produced from cell total RNA or isolated mRNA or genomic DNA or other DNA, or
c) The nucleic acid prep is a chemically or enzymatically synthesized RNA or DNA nucleic acid prep

In certain embodiments of the preceding, a wavelength other than 320 nm is used to detect and quantitate the presence of light scattering substance (LSS) in a nucleic acid solution; a wavelength of equal or greater than 305 nm is used to quantitate the presence of LSS in a nucleic acid solution; a wavelength of between 305 nm and 400 nm, 305 nm and 380 nm, 320 nm and 400 nm, 315 nm and 350 nm, or 320 and 340 nm is used to quantitate the presence of LSS in a nucleic acid solution.

In certain embodiments of the preceding, wavelengths different from 230 nm, 260 nm, 280 nm, and/or 320 nm, are used to determine the nucleic acid prep QQC and/or PQC values; in certain embodiments, the wavelengths are similar to 230 nm, 260 nm, 280 nm, and/or 320 nm; the wavelengths used are within 5%, 4%, 3%, 2%, or 1% of 230 nm, 260 nm, 280 nm, and/or 320 nm.

In particular embodiments of the preceding, the purified nucleic acid prep contains a significant amount of one or more low molecular weight contaminant substances.

In a related aspect, the invention concerns a method for producing improved results for a particular application which uses a nucleic acid prep, where the method includes utilizing one or more embodiments of the preceding aspect to produce improved nucleic acid prep quality characteristic values for the nucleic acid prep, and the nucleic acid prep and improved nucleic acid prep quality characteristic values are used in the particular application which utilizes a nucleic acid prep to produce improved results for the particular application.

In particular embodiments, the particular application is a gene expression analysis assay for determining the number of mRNA or RNA copies per cell for a cell sample, and/or a gene expression comparison analysis assay for determining the fold change or differential gene expression ratio value for a particular gene in compared cell samples, and/or a genomic DNA analysis assay for determining the number of particular gene sequences or nucleotide sequence molecules present in a cell sample or other DNA prep, and/or per cell for a cell sample; and/or another particular application.

Because of the improvement in results for applications using the improved nucleic acid prep quality characteristic values for nucleic acid preps, in a further aspect the invention provides a method for producing improved results for a further particular application which utilize particular application results. The method involves obtaining (e.g., determining) improved particular application results that are produced according to the preceding aspect; and using those improved particular application results in a further particular application which uses particular application results to produce improved further particular application results.

In particular embodiments, the further particular application is or includes a data mining method or process, and/or a systems biology method or process, and/or another further particular application

In additional embodiments of the above aspect or in a further related aspect, the invention provides a method for producing improved results for some other application which utilizes further particular application results, where improved further particular application results produced as described in the preceding aspect are obtained, and utilized in another application which uses further particular application results, producing improved other particular application results.

In particular embodiments, the other particular application includes one or more of:
a) A biological application
b) An industrial application
c) An agricultural application
d) A manufacturing application
e) A basic research application
f) A genetic application
g) A product development application
h) A pharmaceutical application such as drug discover, drug evaluation, drug validity, drug toxicity, drug manufacturing, drug description
i) A toxicology evaluation application
j) A medical or veterinary application.

Additional embodiments will be apparent from the Detailed Description and from the claims.

### DETAILED DESCRIPTION OF THE PREFERRED EMBODIMENTS

In order to assist in understanding the present invention, certain of the terms used herein are described in the table below.

### Glossary

| | |
|---|---|
| Quantitation Quality | The amount of nucleic acid present in the |
| Characteristic (QQC) | nucleic acid preparation or the |
| | concentration of nucleic acid present in |
| | the nucleic acid preparation. Often |
| | measured in micrograms (mcg) or (mcg/ml) |
| | of nucleic acid. |
| Purity Quality | The fraction of the nucleic acid |
| Characteristic (PQC) | preparation which consists of nucleic acid. |
| Integrity Quality | The fraction of nucleic acid molecules in |
| Characteristic (IQC) | the nucleic acid prep which has the |
| | intended or desired nucleotide length or |
| | average nucleotide length. |
| Functional Homogeneity | The functional homogeneity quality |
| Quality Characteristic | characteristic describes the ability of |
| (FHQC) | the nucleic acid molecules in the nucleic |
| | acid preparation to hybridize to the |
| | intended target of the nucleic acid prep. |

### Introduction to the Invention

The underlying basis for the invention is prior art practices which result in either the inaccurate and/or incomplete quality characterization of nucleic acid preps, or a quality characterization of nucleic acid preps which is uninterpretable or of limited interpretability by virtue of not knowing whether the quality characterization is accurate or inaccurate, or complete or incomplete. Such prior art practices are associated with the prior art determination for a nucleic acid prep of quantitative values for the earlier described quality characteristics, the quantitation quality characteristic (QQC), the purity quality characteristic (PQC), the integrity quality characteristic (IQC), and the functional homogeneity quality characteristic (FHQC). These prior art practices, and the solutions to these prior art practices, are discussed below. The solutions to these prior art practices constitute practices of the invention. For simplicity the individual quality characteristic will be referred to as QQC, PQC, IQC, and FHQC.

### Determination of the Quantitation and Purity Quality Characteristics QQC and PQC for a Nucleic Acid Prep.

The QQC for a nucleic acid prep is generally expressed in terms of micrograms (mcg) of nucleic acid per milliliter (ml). This mcg/ml convention will be used herein. The PQC for a nucleic acid prep can be expressed in terms of the fraction of the nucleic acid prep which consists of nucleic acid. Prior art generally refers to the PQC in qualitative terms, such as highly or moderately pure. By far the most widely used method for determining the QQC and PQC for a nucleic acid prep is based on ultraviolet (UV) spectrophotometry. The standard prior art method for determining the QQC for a nucleic acid prep follows. (a) Prepare a solution containing the nucleic acid of interest. (b) Determine an accurate OD260 value for the solution of interest (c) Convert the OD260 value to mcg nucleic acid/ml by using well known conversion factors which relate OD260 values to mcg/ml values for a nucleic acid. It is well known that the quantitative value of such a conversion factor can vary significantly depending on the type of nucleic acid and its state if denaturation, the composition and pH of the measuring solution and temperature(1-4, 10). The quantitative value for the conversion factor should be, but generally is not, determined under the same conditions of temperature, solution composition, and pH, employed for the nucleic acid prep QQC determination, or under conditions which are known to give equivalent results. There is at present no generally accepted solution of known composition and pH which is used to determine the quantitative values for the conversion factors and the QQC values for a nucleic acid prep.

The spectrophotometric OD260 determination is almost always done on a purified nucleic acid prep. It is well known that purified nucleic acid preps commonly contain non-nucleic acid UV absorbing contaminants. As a result it cannot be known from the measured OD260 value that the OD260 value obtained for a measured nucleic acid prep is solely due to the presence of nucleic acid in the measuring solution, and that no non-nucleic acid substances which contribute to the OD260 value are present in the solution. If significant amounts of such non-nucleic acid substances which absorb at 260 nm are present in the measuring solution, then the measured OD260 value will not accurately reflect the presence of only nucleic acids, and will not accurately reflect the concentration of nucleic acids present in the measured solution. Here, the use of an accurate conversion factor to determine the mcg/ml value for the nucleic acid will result in a mcg nucleic acid/ml value for the nucleic acid prep which is significantly over-estimated.

It is well known that the purity of purified nucleic acid preps can and often does, vary greatly, depending on the sample type, treatment, and history, the purification process used, and the types of devices used for the purification and processing of the nucleic acid prep. Significant and varying amounts of one or more of the following substances are known to commonly contaminate purified nucleic acid preps: One or more proteins; small particulate substances of biological origin, such as glycogen; small particulate substances which originate from the purification process or the devices used in the purification process; reagents used in the purification and processing; non-nucleic acid low molecular weight UV absorbing substances; substances which can affect the absorption of nucleic acids at 260 nm, such as salts and buffers; and other biological or non-biological substances (1-14).

It is generally believed that the most common biological contaminant in purified nucleic acid preps is one or more proteins of unspecified type. Most proteins absorb light at 260 nm, but are much less efficient absorbers at 260 nm than are nucleic acids. The measured OD260 value for separate one milligram per ml solutions of nucleic acid and a typical protein, is about 20 and 0.5 respectively (7). Because of this, the presence of a large amount by weight of protein in a purified nucleic acid solution has only a small effect on the accuracy of the OD260 determined QQC value for a nucleic acid prep. The presence in a purified nucleic acid prep of about 80% or 50% protein by weight relative to the nucleic acid present, causes the measured OD260 derived mcg/ml values to deviate from accuracy by about 1.1 fold or 1.04 fold respectively (7).

The presence of small particulate substances in purified nucleic acid preps is also not uncommon (1-4). Such small particulate substances can originate from the source of the nucleic acids such as a biological sample or a chemical or enzymatic nucleic acid synthesis mixture, or from the reagents, processes, or devices, used to purify and process and store the purified nucleic acid prep. The best known small particulate biological substance contaminants are high molecular weight polysaccharides such as glycogen. Such small particulate substance can also originate from pipette tips or glassware or filters used in the purification process, and can also originate in the concentration and resolubilization process, and the freeze thaw process associated with nucleic acid prep storage.

For purified nucleic acid preps which contain protein impurities, there is also the potential to form small particulate molecules composed of complexes of nucleic acid and protein. It is well known in the prior art that small particulate substance or molecules scatter light at all visible and UV wavelengths, including 260 nm (25). It is generally believed that the light scattering associated with such small particulate molecules or substances has the characteristics of, and follows the rules for, Rayleigh light scattering particles. The presence of significant amounts of one or more small particulate substances in a purified nucleic acid prep measuring solution would cause a significant increase in the OD260 value of the solution over that caused by the presence of the nucleic acid in solution. This occurs because a significant fraction of the 260 nm light which is not absorbed by the nucleic acid is scattered away from the spectrophotometer light beam, and is not seen by the instruments light detector. As a result this lost scattered light is measured by the spectrophotometer as if it were being absorbed by the nucleic acid solution. The resulting measured OD260 value is then significantly higher than the OD260 value associated with the nucleic acids in the solution, and converting this measured OD260 value to a mcg nucleic acid/ml value will result in a significant overestimate of the nucleic acid concentration in the solution. For simplification, a small particulate substance will be referred to as a light scattering substance or LSS.

It is not uncommon for a purified nucleic acid prep to contain significant amounts of protein and LSS contaminants. Here, the measured OD260 value for the nucleic acid prep measurement solution will be composed of the sum of the OD260 values associated with the nucleic acid, the protein, and the LSS. Converting the measured OD260 value to a mcg of nucleic acid/ml value will result in a significant overestimate of the nucleic acid concentration for the nucleic acid prep.

The presence of small molecular weight UV absorbing contaminating substances in purified nucleic acid preps is known to be common. Such substances generally originate from the reagents used for the purification process. Examples of such substances include but are not limited to, phenol type compounds, detergent, guanidine thiocyanate, mercaptoethonal, EDTA, salts, and buffers, and others. The presence of significant amounts of such substances in a nucleic acid preparation will cause the OD260 measured and converted value for the mcg of nucleic acid per ml for the nucleic acid prep, to be significantly overestimated. Contamination of a purified nucleic acid prep with one or more of these low molecular weight substances is generally the result of a poorly designed purification protocol or an inadequate purification process technique. Such low molecular weight substances can be readily excluded by the use of the proper protocol and adequate technique.

The presence of small molecular weight substances which can affect the absorption properties of the nucleic acid molecules present in the purified nucleic acid prep is also common. Generally such substances are composed of buffers or ionic salts which are part of the nucleic acid purification and processing and concentration reagents. Generally the presence of such contaminants in a purified nucleic acid prep is the result of a poorly designed purification protocol and/or poor technique. These substances can be readily excluded from the purified prep by the use of a proper protocol and adequate technique.

Generally, the presence of a significant amount of one or more such salt substance in a purified nucleic acid prep causes the nucleic acids in the OD260 measuring solution to absorb significantly less 260 nm light due to the salt-induced increase in nucleic acid secondary structure. This effect is much greater for RNA than DNA. When such a salt effect occurs for an OD260 measurement of a purified nucleic acid prep which contains only nucleic acid and salt, the resulting measured OD260 value converted value for the mcg nucleic acid per ml for the nucleic acid prep is underestimated and inaccurate. This occurs because the OD260 conversion factor value for nucleic acids almost always refers to the OD260 value of a known amount of nucleic acid measured in water or low salt. The presence of contaminant buffer in the purified nucleic acid prep could cause the OD260 measured mcg nucleic acid per ml for the nucleic acid to be significantly inaccurate, and either under- or overestimated. This occurs because the magnitude of the UV absorbance by nucleic acid is pH dependent, and the pH of the solution used to determine the purified nucleic acid prep OD260 value, is different from the pH of the solution used to determine the mcg per OD260 unit conversion factor which is used to obtain the measured mcg of nucleic acid per ml for the nucleic acid prep. Prior art nucleic acid purification practice generally endeavors to produce purified nucleic acid preps which do not contain significant amounts of the above discussed small molecular weight UV absorbing and salt and buffer contaminants. Generally, the purification protocol is designed to eliminate such substances from the purified nucleic acid prep. For the purposes of the description of the present invention, it will herein be assumed that significant amounts of such small molecular weight UV absorbing molecule and salt buffer contaminants are not present in the purified nucleic acid preps discussed, unless otherwise noted.

Prior art is aware that many, if not most, of the generally used prior art nucleic acid purification protocols, produce purified nucleic acid preps which commonly contain significant amounts of protein. Prior art is also aware that LSS contaminants can be present in prior art produced purified nucleic acid preps. Prior art routinely measures the OD260/OD280 ratio value in order to determine whether significant amounts of protein are present in the purified nucleic acid prep of interest. This prior art procedure almost always involves the following. (a) Prepare a solution of the nucleic acid prep of interest. (b) Measure the OD260 and OD280 values for the nucleic acid prep solution of interest. (c) Determine the OD260/OD280 ratio value for the nucleic acid prep solution of interest. (d) Compare the measured OD260/OD280 ratio value of the nucleic acid prep of interest, to the known OD260/OD280 ratio for a relevant nucleic acid preparation which is believed to consist of only nucleic acids. (e) If the OD260/OD280 ratio value for the nucleic acid prep of interest is equal to or greater than the OD260/OD280 ratio value for the “pure” nucleic acid prep, then prior art concludes that the presence of proteins cannot be detected in the nucleic acid prep of interest. (f) If the OD260/OD280 ratio value for the nucleic acid prep of interest is significantly smaller than the OD260/OD280 ratio value for the “pure” nucleic acid prep, then prior art concludes that the nucleic acid prep of interest is contaminated with a significant amount of protein.

Prior art has reported that the OD260/OD280 ratio of a “pure” nucleic acid prep has a value of 2.0 (7). Purified nucleic acid preps with OD260/OD280 ratio values of 1.8 to 2.0 are often produced by the prior art, and are deemed to be of acceptable purity by the prior art. Prior art reports that a purified nucleic acid prep with an OD260/OD280 ratio value of 1.8 contains about 60% by weight of protein, relative to the nucleic acid (7). Prior art is aware that the presence of 60% protein in a purified nucleic acid prep will cause the measured OD260 value for the nucleic acid prep of interest to deviate from accuracy by only 1.04. Prior art then, believes and practices that such a measured OD260 value is not significantly inaccurate, even though significant amounts of protein are present in the purified nucleic acid prep of interest. This prior art belief in the accuracy of measured OD260 value for the purified nucleic acid prep, is not valid if a significant amount of LSS is present in the purified nucleic acid prep. This will be discussed below.

As discussed earlier the presence of significant amounts of LSS in a purified nucleic acid prep will contribute to the measured OD260 and OD280 values for the prep.

Therefore, for a purified nucleic acid prep which contains significant amounts of protein and LSS, (The measured OD260 value for the prep)=(The prep OD260 value due only to nucleic acid)+(The prep OD260 value due only to protein)+(The prep OD260 value due only to LSS). This is illustrated in the hypothetical example of table 1. This hypothetical example represents not uncommon real life purified nucleic acid preps. Table 1 illustrates the effect of the presence of significant amounts of LSS in a purified nucleic acid prep on the validity of the prior art measured OD260/OD280 ratio values for determining the nucleic acid purity of a purified nucleic acid prep, and the validity of the prior art belief and practice that the measured OD260 value for a purified nucleic acid prep accurately measures the preps nucleic acid concentration, even when the measured OD260/OD280 ratio value for the prep is significantly lower than 2.

TABLE 1
EFFECT OF THE PRESENCE OF LSS ON THE PRIOR ART INTERPRETATION OF THE AMOUNT
OF PROTEIN CONTAMINANT IN A PURIFIED NUCLEIC ACID PREP AND THE ACCURACY
OF MEASURED NUCLEIC ACID PREP mcg NUCLEIC ACID PER ml VALUES

| | | | | | | | | | | OD260/ | | |
|---|---|---|---|---|---|---|---|---|---|---|---|---|
| | | | | | Total | | | | Total | OD280 | | OD320 |
| | | OD260 in | | | OD260 | OD280 in | | | OD280 | Ratio | | Value |
| Measured Solution | | Nucleic | | | Value of | Nucleic | | | Value Of | Value Of | | For |
| Composition | | Acid | Protein | LSS | Solution | Acid | Protein | LSS | Solution | Solution | | Solution |
| A. | 50 mcg/ml Nucleic Acid | 1.0 | 0 | 0 | 1.0 | 0.5 | 0 | 0 | 0.5 | | 2 | 0 |
| B. | 50 mcg/ml Protein | 0 | 0.028 | 0 | 0.028 | 0 | 0.05 | 0 | 0.05 | | 0.57 | 0 |
| C. | LSS Only | 0 | 0 | 0.184 | 0.184 | 0 | 0 | 0.136 | 0.136 | | 1.35 | 0.08 |
| D. | 50 mcg/ml Nucleic Acid | 1.0 | 0.028 | 0 | 1.028 | 0.5 | 0.05 | 0 | 0.55 | a) | 1.86 | 0 |
| | (as A) + 50 mcg/ml | | | | | | | | | | | |
| | Protein (as B) | | | | | | | | | | | |
| E. | 50 mcg/ml Nucleic Acid | 1.0 | 0 | 0.184 | 1.184 | 0.5 | 0 | 0.136 | 0.636 | a) | 1.86 | 0.08 |
| | (as A) + LSS (as C) | | | | | | | | | | | |
| F. | 50 mcg/ml Nucleic Acid | 1.0 | 0.028 | 0.184 | 1.212 | 0.5 | 0.05 | 0.136 | 0.686 | b) | 1.77 | 0.08 |
| | (as A) + 50 mcg/ml | | | | | | | | | | | |
| | Protein (as B) + | | | | | | | | | | | |
| | LSS (as C) | | | | | | | | | | | |

Prior Art Interpretations:
a) Prep contains 50% protein by weight (7).
b) Prep contains about 67% protein by weight.

Table 1 uses reference 7 reported nucleic acid and typical protein OD260 and OD280 values for a 1 milligram per ml solution of each, and the OD260/OD280 values of 2 for a pure nucleic acid and 0.57 for a typical pure protein(7). The estimates of the % protein by weight for a particular OD260/OD280 value, also originate from reference 7. One of skill in the art will recognize that these reference 7 values are approximate values and that they are accurate enough for illustration purposes.

Table 1 Rows A and B show the measured OD260 and OD280 values and OD260/OD280 ratio values for separate 50 mcg per ml solutions of pure nucleic acid and pure protein respectively. Table 1 A and B also illustrates that neither protein nor nucleic acid solutions absorb light at 320 nm. Table 1C shows that a solution containing an unspecified amount of LSS gives a significant absorbance value at 260 nm, 280 nm, and 320 nm, and that as expected for Rayleigh scattering, the scattering related OD260 and OD280 values are 2.3 and 1.7 times greater than the OD320 value (25).

Table 1 Row D shows the effect of the presence of 50% protein by weight in a purified nucleic acid prep on the measured OD260 value and the OD260/OD280 ratio value is about 1.86. This would indicate to the prior art that the Table 1D solution contained a significant amount of protein. For this solution the measured OD260 value is 1.028, and prior art would interpret this OD260 of 1.028 value as accurately reflecting the concentration of nucleic acid present in the purified nucleic acid prep solution. In this case, because protein is the only contaminant in the nucleic acid prep solution, the prior art interpretation is valid. Thus, even when the purified nucleic acid prep is contaminated with high amounts of protein, the measured OD260 value for the prep can be validly considered to be accurate for the nucleic acid present in the prep.

Table 1 Row E shows the effect of the presence of LSS in a nucleic acid prep on the measured OD260, OD280, OD320, and OD260/OD280 values, for a nucleic acid prep solution which contains only nucleic acid and the contaminant LSS. For this solution the measured OD320 value is 0.08. Note that this OD320 value of 0.08 is only 0.068 of the OD260 value of 1.184 for the solution. Purified nucleic acid prep solutions with similar or greater OD320/OD260 ratio values are not uncommon. For this solution the OD260/OD280 ratio value is 1.86. Prior art only rarely measures the OD320 value during the process of measuring the OD260 value for a purified nucleic acid prep. On occasion, prior art does measure the OD320 value during this process, but provides no valid guidance as to how the existence of a significant measured OD320 value affects the interpretation of the measured OD260/OD280 ratio value for the prep, or the interpretation of the accuracy of the measured OD260 value in measuring the actual nucleic acid concentration present in the nucleic acid prep.

The standard prior art interpretation of the measured OD260, OD280, OD260/OD280 ratio, and OD320 values for the table 1E nucleic acid follows. (a) The OD260/OD280 ratio value of 1.86 indicates that a significant protein contamination is present in the nucleic acid prep. (b) Because it is known that the presence of a significant amount of protein in the nucleic acid prep solution has little effect on the ability of the measured OD260 value to accurately determine the concentration of nucleic acid present in a nucleic acid prep solution, the measured OD260 value of 1.184 accurately reflects the concentration of nucleic acid present in the solution. (c) The measured OD320 value of 0.08 indicates the presence of an unspecified amount of LSS in the prep. Here the prior art interpretation a) for table 1E is not valid since no protein is present in the nucleic acid prep solution. In addition, prior art interpretation b) is also invalid, and the measured OD260 value significantly overestimates the concentration of nucleic acid which is actually present in the solution.

Note that the wavelength used to detect the presence of LSS in a nucleic acid prep solution can be any wavelength not absorbed by nucleic acids or proteins. Generally this is a wavelength above 305 nm, and the 320 nm wavelength used here is a preferred wavelength, but is only one of many wavelengths which can be employed. Note further that the longer the wavelength employed, the lower the OD value for a solution containing LSS. This occurs because the efficiency of Rayleigh light scattering and light scattering in general is greater for shorter wavelengths of light (25). The light scattering intensity at a short wavelength is greater than the light scattering intensity at a longer wavelength by the factor [1/(short wavelength)⁴]÷[1/(long wavelength)⁴]. This relationship has utility for Rayleigh light scattering particles and the smaller Mie light scattering particles. Thus, an LSS related OD260 value for a solution is about 2.3 times greater than the OD320 value for the same solution, while the LSS related OD280 value for a solution is about 1.7 times larger than the OD320 value for the same solution. Similarly, the LSS related OD260 value for a solution is about 1.35 times larger than the LSS related OD280 value for that same solution. Further, the LSS related OD230 value for a solution is about 3.73 times larger than the OD320 value for the same solution.

The contribution of LSS to the OD230, OD260, and OD280 values of a nucleic acid prep represents the contribution of an unwanted assay variable to these nucleic acid prep OD260 and OD280 values. As a result, the measured prep OD230, OD260, and OD280 values must be corrected or normalized for the presence of the unwanted LSS assay variable signal. The above described LSS related wavelength conversion factors can be used to correct or normalize the measured OD230, OD260, and OD280 values of a nucleic acid prep for the contribution of unwanted LSS related absorbance to these measured values. LSS related absorbance from the measured OD260 value using the relationship, (measured OD260 value normalized for LSS absorbance)=(Measured OD260 value)−(measured OD320 value×2.3). Similarly, (measured OD280 value normalized for LSS absorbance)=(Measured OD280 value)−(Measured OD320 value 1.7). Further, (The measured OD230 value normalized for LSS absorbance) =(Measured OD230 value)−(OD320 value×3.73). For the solution described in table 1E, (The measured OD260 value normalized for the LSS absorbance)=(0.636)−(0.08×1.7)=0.5. Here, the corrected or normalized OD260/OD280 ratio value for the nucleic acid prep solution is equal to 2.0.

From these normalized OD260 and OD280 values, the following can be concluded. (i) There is a significant amount of LSS present in the purified nucleic acid prep, but the LSS related absorbance contribution to the measured OD260 and OD280 values for the prep solution can be normalized for. (ii) The normalized OD260/OD280 ratio value of 2 indicates that the nucleic acid prep does not contain a significant amount of protein contaminant. (iii) The normalized OD260 value accurately reflects the nucleic acid concentration present in the nucleic acid prep. (iv) Relative to the standard prior art practice, these normalized OD260, OD280, and OD260/OD280 values and their interpretation represent invention improved QQC and PQC results, and therefore producing such results is a practice of the present invention. These QQC and PQC results are improved in completeness of normalization, accuracy, interpretability, intercomparability, and utility, relative to prior art produced QQC and PQC results.

Table 1 Row F shows the effect of the presence of significant amounts of both protein and LSS in a nucleic acid prep, on the measured OD260, OD280, OD320, and OD260/OD280 ratio value for a nucleic acid prep solution. For this solution the measured OD320 value is 0.08 and this value of 0.08 is equal to 0.068 of the OD260 value for this solution. Purified nucleic acid prep solutions with similar or greater OD320/OD260 ratio values are not uncommon. For this solution the measured OD260/OD280 ratio value is 1.77. The standard prior art interpretation of these measured OD260, OD280, OD260/OD280 ratio, and OD320 values for this table 1F purified nucleic acid preparation solution follows. (a)The measured OD260/OD280 ratio value of 1.77 indicates that a significant amount of protein is present in the nucleic acid prep. (b) Because it is known that the presence of protein has little effect on the ability of the measured OD260 value to accurately determine the nucleic acid concentration present in a nucleic acid prep solution, the measured OD260 value of 1.212 accurately reflects the concentration of nucleic acid present in the prep solution. (c) The measured OD320 value of 0.08 indicates the presence of a significant amount of unspecified LSS in the prep. Prior art interpretation (a) is valid in this case, as protein is present in the prep. Because prior art does not determine or normalize for the fraction of the measured OD260 and OD280 values which are due to LSS, in reality prior art cannot know that interpretation (a) is valid. Only rarely does prior art determine the OD320 value for a nucleic acid prep solution. Even when prior art determines the OD320 value for a prep, prior art provides no valid guidance as to the use and importance of this value for determining nucleic acid prep QQC and PQC quality characteristics. Interpretation (b) is here invalid, and the measured OD260 value significantly overestimates the concentration of nucleic acid which is actually present in the nucleic acid prep solution.

The above-discussed LSS related wavelength conversion factors can be used to normalize the measured OD260 and OD280 values of table 1F nucleic acid prep solution for the contribution of LSS related absorbance to these values. LSS related absorbance can be removed from the measured OD260 value and measured OD280 value as described earlier. Here, (The normalized OD260 value)=(1.212)−(0.08×2.3)=1.028, and (The normalized OD280 value)=(0.686)−(0.08×1.7)=0.55. The LSS corrected OD260/OD280 value for the nucleic acid prep solution is equal to (1.028/0.55) or about 1.87. From these normalized OD260 and OD280 values for the nucleic acid prep, the following can be concluded about this nucleic acid prep. (i) There is a significant amount of LSS present in the purified nucleic acid prep, but the LSS related absorbance contribution to the measured OD260 and OD280 values for the prep have been normalized for. (ii) The normalized OD260/OD280 ratio value of 1.87 indicates that the nucleic acid prep contains a significant amount of protein contaminant. Absent the LSS normalization, prior art cannot know this. (iii) The normalized OD260 value accurately reflects the concentration of nucleic acid which is present in the nucleic acid prep solution. (iv) Relative to the standard prior art practice, these normalized OD260, OD280, and OD260/OD280 values and their interpretation, represent invention improved quality characteristic results, and therefore producing such results is a practice of the present invention. These improved quality characteristic results are improved in completeness of normalization, accuracy, interpretability, intercomparability, and utility, relative to prior art produced QQC and PQC results. Note that for the table 1F nucleic acid prep solution, knowing that the prep contains a significant amount of contaminant protein constitutes invention improved quality characteristic knowledge. Similarly for a nucleic acid prep solution which has a 260/280 ratio which may indicate the solution is contaminated with protein, knowing that the nucleic acid prep solution does not contain a significant amount of protein contaminant, constitutes invention improved quality characteristics knowledge. Both cases concern improved methods for determining quality characteristics.

As discussed, prior art routinely measures the OD260 and OD280 values for characterizing purified nucleic acid preps. Far less frequently, prior art also measures the OD230, OD260, and OD280 values of purified nucleic acid preps for such a characterization. Prior art indicates that for pure RNA and DNA preps the OD260/OD280 value should be greater than 2 and less than 2.4(1). For simplicity it will herein be assumed that a pure nucleic acid prep has an OD260/OD230 ratio value of 2.4, and an OD260/OD280 ratio value of 2. One of skill in the art will recognize that these values are approximate values and may be different for different purified nucleic acid preps, and that they are accurate enough for illustration purposes.

Generally prior art interprets an OD260/OD230 ratio value of less than 2.4 for a purified nucleic acid prep as being due to contaminants such as phenol, guanidine thiocyanate, other salts, mercaptoethanol, buffer, or protein, while OD260/OD280 ratio values of less than 2.0 are interpreted to be due to contaminant protein. As discussed earlier, it is assumed that the purified nucleic acid preps discussed here have been purified in such a way so that significant amounts of contaminant phenol, guanidine thiocyanate, other salts, mercaptoethanol, buffer, will not be present in the prep.

As discussed earlier, the presence of significant amounts of LSS in a purified nucleic acid prep will contribute to the measured OD230 value for the prep. Therefore, for a purified nucleic acid prep which contains significant amounts of protein and LSS, (The measured OD230 for the value for the nucleic acid prep)=(The prep OD230 value due only to the nucleic acids in the prep)+(The prep OD230 value due only to the protein)+(The prep OD230 value due only to the LSS). This is illustrated in the hypothetical example of table 2. This hypothetical example represents not uncommon real life purified nucleic acid preps. Table 2 illustrates the effect of the presence of significant amounts of LSS in a purified nucleic acid prep on: The validity of the prior art interpretation of the nucleic acid prep purity as measured by the OD260/OD230 ratio value for a purified nucleic acid prep; and the validity of the prior art interpretation concerning whether the measured OD260 value for a nucleic acid prep accurately reflects the nucleic acid concentration which is present in the purified nucleic acid prep.

TABLE 2
EFFECT OF THE PRESENCE OF LSS ON THE PRIOR ART INTERPRETATION OF THE AMOUNT
OF PROTEIN CONTAMINANT IN A PURIFIED NUCLEIC ACID PREP AND THE ACCURACY
OF MEASURED NUCLEIC ACID PREP mcg NUCLEIC ACID PER ml VALUES

| | | OD260 | | | | OD230 | OD230 | | | | |
|---|---|---|---|---|---|---|---|---|---|---|---|
| | | In | OD260 | OD260 | | In | In | OD230 | | OD260/ | |
| | | solution | In | In | Total | solution | solution | In | Total | OD230 | OD320 |
| Measured | | Due to | solution | solution | OD260 | Due to | Due to | solution | OD230 | Ratio | Value |
| Solution | | Nucleic | Due to | Due to | Value of | Nucleic | Protein | Due to | Value of | Value of | For |
| Composition (a) | | Acid | Protein | LSS | Solution | Acid | (b) | LSS | Solution | Solution | Solution |
| A. | 50 mcg/ml Nucleic Acid | 1.0 | 0 | 0 | 1.0 | 0.42 | 0 | 0 | 0.42 | 2.4 | 0 |
| B. | 50 mcg/ml Protein | 0 | 0.028 | 0 | (b) 0.028 | 0 | 0.26 | 0 | (b) 0.26 | (b) 0.107 | 0 |
| C. | LSS Only | 0 | 0 | 0.184 | 0.184 | 0 | 0 | 0.296 | 0.296 | 0.62 | 0.08 |
| D. | 50 mcg/ml Nucleic Acid | 1.0 | 0.028 | 0 | 1.028 | 0.42 | 0.26 | 0 | 0.68 | 1.51 | 0 |
| | (As A) + 50 mcg/ml | | | | | | | | | | |
| | Protein(As B) | | | | | | | | | | |
| E. | 50 mcg/ml Nucleic Acid | 1.0 | 0 | 0.184 | 1.184 | 0.42 | 0 | 0.296 | 0.716 | 1.65 | 0.08 |
| | (As A) + LSS (As C) | | | | | | | | | | |
| F. | 50 mcg/ml Nucleic Acid | 1.0 | 0.028 | 0.184 | 1.212 | 0.42 | 0.26 | 0.296 | 0.976 | 1.24 | 0.08 |
| | (As A) + 50 mcg/ml | | | | | | | | | | |
| | Protein (As B) + | | | | | | | | | | |
| | LSS (as C) | | | | | | | | | | |

(a) These solution compositions are identical to those in Table 1.
(b) These are approximate values for bovine serum albumin. Different proteins can have different values (26).

Table 2A and B show measured OD260 and OD230 values and OD260/OD230 ratio values for separate 50 mcg per ml solutions of pure nucleic acid and pure protein. Table 2C shows that an LSS containing solution identical to the one in table 1C gives a significant absorbance value at 260 nm, and 230 nm, and that as expected, the light scattering related absorbance at 230 nm is 3.73 and 1.6 times greater than at 320 nm and 260 nm respectively.

Table 2D shows the effect of the presence of 50% by weight protein in a purified nucleic acid prep on the measured OD260 value and the OD260/OD280 ratio value for the nucleic acid prep solution. For this solution measured OD260/OD230 ratio value is 1.51. Prior art would interpret this to mean that the nucleic acid prep was significantly contaminated with a protein. This prior art interpretation is valid. The measured OD260 value for the nucleic acid prep is 1.028, and prior art would interpret this measured OD260 value as accurately reflecting the concentration of nucleic acid present in the prep. This prior art interpretation is valid. A significant measured OD320 value was not detected for this solution. Prior art would interpret this to mean that a significant amount of LSS is not present in the solution. This is a valid interpretation.

Table 2E shows the effect of the presence of LSS in the purified nucleic acid prep on the measured OD260, OD230, OD320, and OD260/OD280 ratio values for a prep containing only nucleic acid and LSS. For this solution the measured OD230, OD260, OD320, and OD260/OD280 ratio values are 0.716, 1.184, 0.08, and 1.65 respectively. Prior art only rarely measures the OD320 value for a nucleic acid prep, and provides no valid guidance as to how the existence of significant measured OD320 values affects the interpretation of the measured OD260/OD320 ratio value, and the accuracy with which the measured OD260 value reflects the actual nucleic acid concentration present in a nucleic acid prep. The standard prior art interpretation of the table 2E measured OD230, OD260, OD320, and OD320, and OD260/OD230 ratio values follows. (a) The OD260/OD230 ratio value of 1.65 indicates that the nucleic acid prep contains significant amounts of one or more contaminants. However, the identity of the contaminants cannot be determined from the ratio value. This interpretation is correct. (b) The measured OD260 value of 1.184 is generally believed to accurately reflect the concentration of the nucleic acid in the prep. Depending on the nature of the nature of the contaminant this may or may not be a valid interpretation. For example, as discussed above if the contaminant is protein then the interpretation is correct or nearly correct, and if the contaminant is LSS the interpretation is incorrect. (c) The measured OD320 value of 0.08 indicates the presence of significant amounts of LSS in the nucleic acid prep. This interpretation is correct.

As discussed, the table 2E measured OD230, OD260, and OD260/OD230 ratio values, can be corrected or normalized for the LSS related absorbance. The LSS corrected values for the table 2E OD230, OD260, and OD260/OD230 ratio values are, 1.0, 0.42, and 2.4 respectively. From these LSS normalized OD230, OD260, and OD260/OD230 ratio values, the following can be concluded.
(i) The normalized OD260/OD230 ratio value of 2.4 indicates that the nucleic acid prep does not contain a significant amount of other non-LSS contaminants. The combination of the table 1E LSS normalized OD260/OD280 ratio value of 2.0, and this table 2E LSS normalized OD260/OD230 ratio value of 2.4 strongly supports the conclusion that no other non-LSS contaminants in significant quantities are present in the nucleic acid prep.
(ii) The normalized OD260 value accurately reflects the nucleic acid concentration present in the nucleic acid prep. (iii) Relative to standard prior art practice, these normalized OD260, OD230, and OD260/OD230 ratio values, and their interpretation, represent invention improved quality characteristic results, and therefore producing such results is a practice of the present invention. These QQC and PQC results are improved in completeness of normalization, accuracy, interpretability, intercomparability, and utility, relative to prior art produced quality characteristic results.

Table 2F shows the effect of the presence of significant amounts of contaminating protein and LSS in a purified nucleic acid prep, on the measured OD230, OD260, OD320, and OD260/OD230 ratio values. For this solution the measured OD230, OD260, OD320, and OD260/OD230 ratio values are 0.976, 1.212, 0.08, and 1.24 respectively. The standard prior art interpretation of these table 2F values follow. (a) The OD260/OD230 ratio value of 1.24 indicates that the nucleic acid prep contains very significant amounts of one or more contaminants. However, the identity of the contaminant cannot be determined from the ratio value. (b) The measured OD260 value if 1.212 is generally believed by the prior art to be an accurate measure of the concentration of nucleic acid present in the prep. Depending on the nature of the contaminant this interpretation may or may not be correct or nearly correct. For example as discussed above, if the contaminant is protein then the interpretation is correct or nearly correct. (c) The measured OD320 value of 0.08 indicates the presence of significant amounts of LSS in the nucleic acid prep solution. This interpretation is correct, but prior art gives no guidance as to the effect of the measured OD320 value for the prep on the overall interpretation of the measured OD260, OD280, and OD260/OD280 ratio values for the prep. As discussed, because of the existence of this significant OD320 value, the measured OD260 value for the prep significantly overestimates the concentration of the nucleic acid present in the nucleic acid prep.

As discussed, the table 2F measured OD230, OD260, and OD260/OD230 ratio values, can be corrected or normalized for the LSS related absorbance. The LSS corrected values for the table 2F OD230, OD260, and OD260/OD230 ratio values are, 1.28, 0.68 and 1.51 respectively. From these LSS normalized OD230, OD260, and OD260/OD230 ratio values, the following can be concluded. (i) The normalized OD260/OD230 ratio value of 1.51 indicates that significant amounts of contaminant are present in the nucleic acid prep. The combination of the table 1F LSS normalized OD260/OD280 ratio value of 1.86 and the table 2F LSS normalized OD260/OD230 value of 1.51 supports this conclusion. (ii) The normalized OD260 value for the nucleic acid prep accurately reflects the concentration of nucleic acid present in the nucleic acid sample solution. (iii) Relative to standard prior art practice, these normalized OD230, OD260, and OD260/OD230 ratio values and their interpretation represent invention improved QQC and PQC results, and therefore producing such results is a practice of the invention. These QQC and PQC results are improved in completeness of normalization, accuracy, interpretability, intercompatibility, and utility, relative to prior art produced QQC and PQC results.

As discussed earlier, for simplicity the tables 1 and 2 illustrations assumed that OD260/OD230 and OD260/OD280 ratio values of 2.4 and 2.0 respectively, are characteristic of contaminant-free pure nucleic acid preps. In real life these ratio values are different for different types of nucleic acid, e.g. RNA or DNA, and can be different for different types of RNA or DNA, depending on the secondary structure and base composition of the RNA or DNA. Similarly, for simplicity the tables 1 and 2 illustrations assumed that a 1 mg/ml solution if nucleic acid has an OD260 value of 20. In real life the OD260 value per mg of nucleic acid are different for different types of nucleic acid, e.g. RNA or DNA, and are different for different forms of RNA or DNA, e.g. double and single strand RNA or DNA, and can be different for different types of RNA or DNA depending on the base composition and secondary structure of the nucleic acids, and the composition and pH of the measuring solution used.

In this context, in order to determine accurate OD260/OD230 and OD260/OD280 ratio values, and an accurate value for the OD260 units per mg of nucleic acid for a particular RNA or DNA, it is necessary to be able to obtain pure RNA or DNA of the particular type of interest. For a biological cell sample RNA or DNA of interest or an enzymatically or chemically synthesized RNA or DNA of interest, one or another of existing prior art methods for obtaining highly purified RNA or DNA can be used to obtain such pure RNA or DNA. The OD260/OD230 and OD260/OD280 ratio values, and the OD260 conversion value for a particular “pure” nucleic acid prep, and the OD230, OD260, OD280, and OD320 values for an unknown nucleic acid prep should be determined in a standard measuring solution of known composition and pH.

A variety of prior art methods are available for producing highly purified nucleic acid preps. Certain of these methods are suitable for producing highly purified nucleic acid preparation which do not contain significant amounts of contaminants (18, 19). As a general rule, small molecular weight contaminants are easily eliminated from a nucleic acid prep. As an example, substances such as phenol, guanidine thiocyanate, mercaptoethanol, detergents, buffers, salts, and others, can be readily eliminated by careful laboratory procedures which include an appropriate method for washing a precipitated nucleic acid prep. Such methods are easily implemented and commonly used (1-11).

Higher molecular weight contaminants which often co-purify and/or co-precipitate with RNA or DNA are more difficult to eliminate. These contaminants include certain proteins and polysaccharides. Many, if not most of the most commonly used prior art nucleic acid purification methods do not eliminate these higher molecular weight contaminants from the purified nucleic acid preps. These higher molecular weight contaminants are generally more difficult to eliminate, and the methods which can ensure the absence of such contaminants are more complex and time consuming than the commonly used nucleic acid purification methods. Perhaps the most effective method for ensuring the absence of significant amounts of essentially all potential contaminants in a purified nucleic acid prep, is a combination of preparative centrifugation and dialysis methods (18, 19). Such centrifugation methods include various denaturing and non-denaturing density gradient centrifugation methods which are well known. Other methods such as denaturing size exclusion chromatography can also be effective for this purpose.

As discussed above, most of the commonly used prior art nucleic acid prep purification methods will, when properly done, eliminate low molecular weight contaminants from the produced purified nucleic acid prep, but do not eliminate certain high molecular weight contaminants such as proteins or polysaccharides from the produced purified nucleic acid prep. Therefore, it is not uncommon for such prior art purified nucleic acid preps to contain significant amounts of protein and/or polysaccharide contaminants. The presence of significant amounts of one or more contaminants is generally undesirable and can make it difficult to assess the nucleic acid concentration and purity of a nucleic acid prep. It is important to obtain an accurate assessment of the concentration of nucleic acid present in a purified nucleic acid prep. Different nucleic acid preps are often compared for a nucleic acid prep application, and it is important to be able to compare known amounts of such nucleic acids. As an example, standard gene expression comparison assays attempt to compare equal quantities of nucleic acids. It is also important to have a measure of the purity of compared nucleic acid preps. For example, the presence of contaminants in a nucleic acid prep can negatively affect the in vitro enzymatic synthesis of labeled and unlabeled cDNA and cRNA from RNA and DNA, and the amplification efficiency of the polymerase chain reaction. Ideally, the nucleic acid preps used in all nucleic acid prep applications should be contaminant free. In reality, it is common for such purified nucleic acid preps to be significantly contaminated.

The illustrations of tables 1 and 2 and the various descriptions and discussions of the practice of the invention using these illustrations, describe the general rationales and processes involved in the practice of the invention. From these general descriptions and discussions of the practice of the invention, one of skill in the art will recognize the method for practicing the invention for a particular purified nucleic acid prep. Further, one of skill in the art will recognize that wavelengths longer than 320 nm can be utilized to practice the invention, and that the wavelength chosen to detect the presence of LSS can vary for different nucleic acid preps. For example certain linkers which are used to covalently attach a chemical entity such as biotin, or a fluorescent molecule to RNA, cRNA, DNA, or cDNA molecules, absorb significantly at 320 nm, but do not absorb at certain longer wavelengths. For these and other similar situations, one of skill in the art will recognize that it is preferable to utilize a longer than 32 onm wavelength to detect the presence of LSS.

Characteristics of the preferred practice of the invention for determining the QQC for a purified nucleic acid prep follow. (a) In order to facilitate the detection of low levels of LSS the OD260 of the measured nucleic acid prep solution should be as high as is practical for the spectrophotometer used. (b) A standard optimized absorbance measuring solution of known composition and pH should be used for measuring the OD260, OD280, and OD320 values for different purified nucleic acid preps. It may be desirable to use different solutions for RNA and DNA. (c) Contaminant SAS, salts, and buffers should be essentially eliminated from the nucleic acid prep. A variety of prior art procedures are available for accomplishing this. (d) For each nucleic acid prep replicate dilutions should be made, and replicate measurements for each dilution should be done. (e) To confirm the light scattering nature of a significant OD320 value for a nucleic acid prep solution, also measure the OD400 value for the solution. The OD320 value should be about 2.4 times greater than the OD400 value. (f) For each nucleic acid prep the OD230, OD260, OD280, and OD320 should be determined.

### Determination of Integrity Quality Characteristic (IQC)

The integrity quality characteristic (IQC) is expressed in terms of a measure of the fraction of the nucleic acid molecules present in the nucleic acid prep which has the desired or intended nucleotide length or average nucleotide length. Such nucleic acid preps include, for example, the following: complex total RNA, isolated mRNA, or total DNA preps from prokaryotic or eukaryotic cells; RNA or DNA preps from viruses; complex in vitro enzymatically synthesized cDNA or cRNA preps from cell total RNA or cell isolated mRNA; complex in vitro enzymatically synthesized genomic DNA preps from cell DNAs; in vitro enzymatically synthesized cDNA or cRNA preps or genomic DNA preps from viruses; in vitro enzymatically synthesized particular gene mRNA preps or particular gene or nucleotide sequence DNA preps; particular chemically synthesized RNA or DNA sequence preps.

The simplest nucleic acid preps to characterize are those which are intended to represent a homogeneous population of nucleic acid molecules which consist of nucleic acid molecules which all have the same nucleotide length, nucleotide sequence, and nucleotide composition. Here, an IQC value of 1 would indicate that all of the nucleic acid molecules in the nucleic acid prep have that same nucleotide length, while an IQC value of 0.5 would indicate that 50% of the nucleic acid molecules in the prep have the intended nucleotide length. These simplest to characterize nucleic acid preps include chemically synthesized nucleic acid preps of virtually all kinds, in vitro enzymatically synthesized particular gene mRNA preps, and particular gene or nucleotide sequence enzymatically synthesized DNA preps. The intended nucleotide lengths of these various prior art produced simple nucleic acid preps range from about 4 nucleotides long to thousands of nucleotides long. A variety of prior art methods are available for determining the quantitative IQC value for these simple RNA and DNA preps. These methods include, but are not limited to: native and denaturing gel electrophoresis; native and denaturing density gradient sedimentation; native and denaturing size exclusion column separation; various forms of mass spectroscopy; various forms of native and denaturing capillary electrophoresis. For characterizing the single strand forms of RNA or DNA it is preferable to utilize a denaturing characterization method. For characterizing double strand RNA or DNA molecules both native and denaturing characterization methods should be used.

Chemically synthesized nucleic acid preps are generally intended to be composed of a homogeneous population of nucleic acid molecules, all of which have the same nucleotide length. Prior art often determines a quantitative value for the IQC of a chemically synthesized nucleic acid prep. This is usually obtained by gel electrophoresis, capillary gel electrophoresis, high pressure liquid chromatography, or mass spectroscopy methods. Such IQC determinations are generally accurate for shorter nucleic acid molecules less than about 50-60 nucleotides long, but are less accurate for longer molecules. These same methods can be used to obtain quantitative values for the longer molecules which represent the fraction of chemically synthesized molecules which have nearly the same nucleotide length as the intended nucleotide length.

Prior art commonly produces RNA or mRNA for a particular gene, or DNA for a particular DNA sequence of defined length, by in vitro enzymatic synthesis. The mRNAs are generally polyadenylated. The intended nucleotide length of such RNA or DNA molecules generally ranges from about 20 to thousands of nucleotides long but can be shorter. The IQC for such synthesized RNA or DNA preps is usually measured by native or denaturing gel electrophoresis or capillary gel electrophoresis. Such prior art determined IQC measurements almost always represent a semi-quantitative estimate of the fraction of the synthesized RNA or DNA prep which consists of RNA or DNA molecules of nearly the intended nucleotide length. Many such prior art IQC measurements are obtained using the Agilent bioanalyser capillary gel electrophoresis system. The analysis condition for this system is non-denaturing.

Prior art commonly produces complex RNA or genomic DNA preps from cell samples. These include purified cellular RNA and isolated mRNA preps, and purified genomic DNA preps. Such RNA or DNA preps consist of a complex mixture of many different particular gene RNA molecules, or many genomic DNA molecules with different sequences. A purified genomic DNA prep generally consists of a population of DNA molecules with a roughly Gaussian distribution of nucleotide lengths. Generally, it is desired by the investigator that a purified DNA prep have an intended average DNA molecule nucleotide length and DNA molecule nucleotide length distribution. For such a DNA prep then, the IQC measurement indicates whether the DNA molecule population of the purified genomic DNA prep has the intended average nucleotide length and nucleotide length distribution.

While purified total cell RNA is composed of the RNA transcripts from a large number of different genes, about 80-90% of the total RNA consists of two ribosomal RNA subunits, one large and one small. These small and large ribosomal RNA subunit molecules have different and known nucleotide lengths, and are often used as internal markers to judge the integrity of the total RNA prep. Here, the prior art measures the IQC in terms of: whether ribosomal RNA molecules of the intended or correct natural nucleotide length can be detected in the total RNA; and whether the ratio of the amount of large and small ribosomal RNA molecules present in the total RNA prep is the intended or natural ratio. In other words prior art measures the IQC for a purified cell total RNA prep by determining the following. (i) Are there any detectable undegraded large and small ribosomal RNA subunit molecules present in the total RNA prep? (ii) Is the ratio of undegraded large and small ribosomal molecules the intended ratio? The answers to (i) and (ii) provide a roughly quantitative measure of the total RNA IQC (12).

A mammalian cell purified mRNA prep is produced from purified total RNA and contains little ribosomal RNA. The mRNA prep consists of mRNA transcript molecules from a large number of different cell genes. A mRNA prep which is undegraded is composed of a population of mRNA molecules which have the same nucleotide sequence length which they had in the intact cells. The nucleotide lengths of different undegraded mRNA molecules in the prep range from 200 to thousands of nucleotides. For a typical mammalian cell undegraded mRNA prep, the average mRNA molecule nucleotide length is about 1800 nucleotides, and the nucleotide length distribution is broad. Generally it is desired that a purified mammalian cell mRNA prep have an intended average nucleotide length of about 1800 and a distribution which is the same as an undegraded mRNA prep from the cells of interest. For a purified mRNA prep then, the IQC measurement indicates the purified mRNA molecule population has an average nucleotide length of about 1800 nucleotides, and a nucleotide sequence distribution which is essentially the same as that for undegraded cell mRNA prep. The greater the deviation of the mRNA prep from the undegraded average size and distribution, the lower the integrity of the mRNA prep.

A similar situation to the above described purified mammalian mRNA prep IQC determination, exists for purified unlabeled or labeled cDNA or cRNA preps produced from cell mRNA or total RNA preps. For such a purified cell mRNA derived cDNA or cRNA prep, the IQC indicates whether the cell cDNA or cRNA prep molecules have an average nucleotide length of about 1800 nucleotides, and a nucleotide length distribution which is essentially the same as the cell RNA which it was produced from. The greater the deviation of the cDNA or cRNA prep from the undegraded nucleotide length and distribution, the poorer the integrity of the prep. While the above discussion has been in terms of mammalian cell mRNA, cDNA and cDNA purified preps, the basic method for determining and evaluating the IQC applies to cell mRNA, cDNA, and cRNA preps of all kinds. The determination of the average nucleotide length and distribution of nucleotide lengths for cell mRNA, cDNA, or cRNA preps of all kinds is commonly done using native or denaturing gel electrophoresis or capillary gel electrophoresis.

### Determination of the Functional Homogeneity Quality Characteristics (FHQC) of Purified Nucleic Acid Preps

The FHQC of a purified nucleic acid prep is expressed in terms of the fraction of the purified nucleic acid prep which can hybridize with the nucleic acid preps intended target complementary nucleic acid molecule. Therefore, in order to determine the FHQC value for a nucleic acid prep, an intended target complementary nucleic acid prep must be available. Further, in order to determine a valid FHQC value for a nucleic acid prep, a hybridization assay method must be used which will allow the quantitative detection and determination of all of the nucleic acid molecules in a prep which are capable of hybridization with the intended target. Prior art rarely attempts to determine the quantitative FHQC value for a purified nucleic acid prep.

The simplest nucleic acid preps to characterize are those which are intended to represent a homogeneous population of nucleic acid molecules compromised of nucleic acid molecules which all have the same nucleotide length, nucleotide sequence, and nucleotide composition. Here an FHQC value of 1 indicates that all of the nucleic acid molecules in the purified nucleic acid prep can hybridize to the intended target nucleic acid, while an FHQC value of 0.5 indicates that only 0.5 of the nucleic acid prep molecules can hybridize to the intended target nucleic acid. These simplest to characterize nucleic acid preps include the chemically synthesized nucleic acid preps of virtually all kinds, and in vitro enzymatically synthesized particular gene RNA preps or particular nucleotide sequence DNA preps of all kinds. Herein, such a nucleic acid prep which is intended to represent a homogeneous population of nucleic acid molecules is termed a simple nucleic acid prep.

Generally it is possible to produce a highly purified complementary target simple nucleic acid prep with an intended nucleotide length of up to about 100-200 nucleotides long. Such a simple complementary target nucleic acid molecule prep can be readily produced by chemical synthesis and should be highly purified for the intended length nucleic acid molecules. Prior art routinely produces such purified chemically synthesized nucleic acid preps. Complementary target nucleic acid molecule preps which are longer than 100 nucleotides long are routinely produced in vitro enzymatic methods. Single or double strand nucleic acid molecules which range from 50-100 to thousands of nucleotides long can be produced by these methods. Both RNA and DNA target molecules can be produced by these methods, which include various polymerase chain reaction (PCR) and other DNA amplification methods (1, 4, 27), DNA cloning and RNA synthesis methods (20-24), and RNA synthesis promoter methods for producing cRNA (4, 12-15). Preferably such simple target nucleic acid preps should be highly purified and well characterized.

A general protocol for the determination if the FHQC value for a simple nucleic acid prep follows. (a) Produce a simple intended nucleic acid prep of interest which is directly labeled with a signal generation molecule or complex. A directly labeled nucleic acid molecule has the signal generating molecule attached directly to the nucleic acid. A variety of such signal molecules are available for this purpose, including radioactive and fluorescent molecules (4). One of skill in the art will be aware that an indirect label molecule can be directly attached to a nucleic acid molecule during production. There are many such prior art indirect label molecules, and these include biotin and various haptens (4). Such indirect label molecules allow the direct attachment of a signal generating molecule complex to the nucleic acid molecule after hybridization. The preferred labeled nucleic acid prep is a directly labeled radioactive nucleic acid prep. (b) Produce the simple complementary unlabeled target nucleic acid prep of interest. (c) Add a known mole amount of the labeled simple nucleic acid prep of interest to the chosen hybridization solution. (d) To the same hybridization solution add an amount of unlabeled complimentary nucleic acid target which is known to be in significant mole excess over the amount of labeled simple nucleic acid present. Preferably, such molar excess should be between two and tenfold. (e) Place the hybridization solution under the chosen hybridization conditions and incubate long enough to obtain the maximum hybridization. Preferably the hybridization conditions should be stringent hybridization conditions. (f) Assay the hybridization solution and determine the fraction of the labeled nucleic acid prep which is capable of hybridizing with the unlabeled complementary nucleic acid prep. This fraction is a quantitative measure of the FHQC. A variety of prior art hybridization assay methods can be used to accomplish the FHQC measurement. Such methods include hydroxyapatite and nuclease protection methods (1, 4, 28)

Many nucleic acid preps are complex and consist of a heterogeneous mixture of nucleic acid molecules which are of different nucleotide length and nucleotide sequence. All prokaryotic and eukaryotic cell purified total RNA or mRNA preps, and purified genomic DNA preps are complex nucleic acid preps. In theory, the determination of the FHQC value for such a complex nucleic acid prep is possible and can be done as described above. Practically, this can be done for only for purified cell genomic DNA and RNA preparations which have relatively low nucleotide sequence complexity. This includes most prokaryote and some eukaryote purified cell genomic DNAs. Currently it would be very difficult to determine an FHQC value for a purified eukaryotic cell total RNA or isolated mRNA prep. Such a determination for a purified prokaryotic cell total RNA prep or isolated mRNA prep is possible.

### Use of Nucleic Acid Prep Quality Characteristic Values and Combinations of Nucleic Acid Prep Quality Characteristic Values Which are Invention Improved.

Generally, prior art produces a nucleic acid prep in order to use the prep for particular application which utilizes a nucleic acid prep. Prior art believes and practices that accurate knowledge of one or more of the quality characteristics of the nucleic acid prep is necessary in order to obtain accurate and interpretable results for the particular application which the nucleic acid prep is used for. As an example, for gene expression analysis and gene expression comparison analysis of all kinds, prior art routinely attempts to determine the QQC and PQC values for the analyzed nucleic acid preps, and less frequently attempts to determine the IQC for the analyzed nucleic acid preps. These nucleic acid quality characteristic values are determined because prior art believes it is necessary to know accurate values for the quality characteristics of a nucleic acid prep in order to obtain accurate and interpretable results for the particular application which uses the nucleic acid prep.

The practice of the invention produces nucleic acid prep quality characteristic values and combinations of nucleic acid quality characteristic values which are significantly improved in accuracy, interpretability, reproducibility, intercomparability, and utility, relative to prior art produced nucleic acid prep quality characteristic values, and combinations of nucleic acid prep quality characteristic values. Therefore, the use of one or more invention improved nucleic acid prep quality characteristic values for a particular application which uses the nucleic acid prep, produces invention improved particular application result values which are improved in accuracy, reproducibility, intercompatibility, interpretability, and utility. The production of such improved particular application results is then, a practice of the present invention.

Prior art produced particular application results are often used as part of a further particular application, to obtain further particular application results. The practice of the invention produces nucleic acid prep quality characteristic value results, and particular application results, which are significantly improved relative to prior art produced nucleic acid prep quality characteristic value results and particular application results. The use of such improved particular application results in a further particular application, produces further particular application results which are improved in accuracy, interpretability, intercomparability, reproducibility, and utility, relative to prior art produced further particular application results, by virtue of being produced using invention improved nucleic acid prep quality characteristic results and particular application results. Therefore producing such improved further particular application results is a practice of the invention. Examples of such particular application results are gene expression assay measured mRNA abundance values and gene expression comparison assay measured particular gene fold change ratio results (4). Examples of further particular application results are data mining process results and systems biology results which utilize gene expression analysis and gene expression comparison analysis results.

Prior art produced further particular application results are often used for other purposes such as drug discovery, drug validation, drug evaluation, drug manufacturing, drug toxicology, drug prescription, general toxicology evaluation, and many other purposes. Here, the use of the invention improved further particular application results for other purposes results in the production of improved other purpose results, and is a practice of the present invention.

### EXAMPLES

Various examples of the practice of the invention are listed below.

### Example 1

### A Preferred Practice

This aspect of the invention is practiced as follows.
(a) Produce a purified nucleic acid prep which does not contain significant amounts of lower molecular weight UV absorbing contaminant molecules.
(b) For the purified nucleic acid prep measure the absorbance values at 230 nm, 260 nm, 280 nm, and at 320 nm or a longer wavelength, and do this in a solution of known composition and pH.
(c) Normalize the measured OD230, OD260, and OD280 values for the measured absorbance at 320 nm or alternate longer wavelength.
(d) From the normalized OD260/OD280 ratio values for the nucleic acid prep, determine the PQC value for the prep.
(e) From the normalized OD260 value for the nucleic acid prep determine the concentration of nucleic acid present in the nucleic acid prep. The conversion factor used to convert the OD260 value to a mcg nucleic acid per ml value should be obtained using the same measuring solution of known composition and pH, and close to the same measurement temperature, or equivalent conditions of measurement solution composition, pH, and temperature.

### Example 2

As example 1 except that either the OD230 value or the OD280 value is not determined, and therefore either the OD260/OD230 ratio value or the OD260/OD280 ratio value for the nucleic acid prep cannot be used to determine the PQC.

### Example 3

As example 1 except that neither the OD230 value nor the OD280 value are determined. Therefore neither the OD260/OD230 ratio value nor the OD260/OD280 ratio value can be used to evaluate the PQC and a measure of the PQC cannot be determined for the nucleic acid prep.

### Example 4

As example 1 or 2 or 3, except that either, or both, of the IQC and FHQC values are also determined for the nucleic acid prep of interest.

### Example 5

As example 1 or 2 or 3 or 4 wherein the nucleic acid prep is a chemically or in vitro enzymatically synthesized RNA or DNA prep.

### Example 6

As example 1 or 2 or 3 or 4 wherein the nucleic acid prep is a cell total RNA or isolated mRNA, or genomic DNA prep.

### Example 7

As example 1 or 2 or 3 or 4 wherein the nucleic acid prep is cRNA or cDNA or amplified genomic DNA produced from cell total RNA, isolated mRNA or genomic DNA.

### Example 8

As examples 1 thru 7 except that a wavelength other than 320 nm is used to detect and quantitate the presence of significant amounts of LSS in a nucleic acid prep.

### Example 9

As examples 1 thru 8 wherein wavelengths different from but similar to 230 nm, 260 nm, 280 nm, and 320 nm, are used to determine the QQC and PQC values for a nucleic acid prep.

### Example 10

As examples 1 thru 9 except that the purified nucleic acid prep contains a significant amount of one or more low molecular weight contaminant substances.

### Example 11

This aspect if the invention is practiced as follows. (a) Produce one or more invention improved nucleic acid prep quality characteristic values. (b) Use the nucleic acid prep and the invention improved quality characteristic values in a particular application which utilizes a nucleic acid prep. (c) Produce the improved particular application result.

### Example 12

As example 11 where the improved particular application result is a gene expression assay determined abundance value or copy per cell value for a particular gene mRNA in a cell sample.

### Example 13

As example 11 where the improved particular application result is a gene expression comparison assay determined differential gene expression ratio value or fold change value for a particular gene mRNA in compared cell samples.

### Example 14

This aspect of the invention is practiced as follows. (a) Produce one or more invention improved nucleic acid prep quality characteristic values. (b) Use the nucleic acid prep and the invention improved quality characteristic values in a particular application which utilizes a nucleic acid prep to produce invention improved particular application results. (c) Use the invention improved particular application results in a further particular application which utilizes particular application results to produce an improved particular application result.

### Example 15

As example 14 where the further particular application is a data mining process application.

### Example 16

As example 15 where the further particular application is a systems biology process application.

### Example 17

This aspect of the invention is practiced as follows. (a) Produce one or more invention improved nucleic acid prep quality characteristic values. (b) Use the nucleic acid prep and the invention improved quality characteristic values in a particular application which utilizes a nucleic acid prep to produce invention improved particular application results. (c) Use the invention improved particular application results in a further particular application which utilizes particular application results, to produce invention improved further particular application results. (d) Use the invention improved further particular application results in some other application which uses further particular application results, to produce invention improved other application results.

### Example 18

As example 17 where the other application result is: a drug discovery result; and/or a biomarker discovery result; a drug validation result; a drug toxicity result; a drug evaluation result; a drug manufacturing result; a drug prescribing result; another drug development related result; a non-drug related toxicology result; an industrial related result; an agricultural related result; some other result.

### REFRENCES

1. Farrel, R. E. RNA Methodologies a Laboratory Guide For Isolation And Characterization. Third Ed. Chapter 8. Elseveier (2005)
2. Adams, D. S. LAB Math. A Handbook of Measurements, Calculations, And Other Quantitative Skills For Use At The Bench. Chapter 5. Cold Spring Harbor Laboratory Press (2003)
3. Stephenson, F. H. Calculations For Molecular Biology And Biotechnology. A Guide To Mathematics In The Laboratory. Chapter 5 Academic Press (2003)
4. Botwell, D. and Sambrook, J. DNA Microarrays. A Molecular Cloning Manual. Cold Spring Harbor Laboratory Press (2003)
5. Stulnig, T. M. and Amberger, A. Exposing Contaminating Phenol In Nucleic Acid Prepparations. Biotechniques 16(3):402-404(1994)
6. Huberman, J. A. Importance Of Measuring Nucleic Acid Absorbance At 240 nm As Well As At 260 and 280 nm. Biotechniques 18(4):636(1995)
7. Glasel, J. A. Validity Of Nucleic Acid Purities Monitored By 260 nm/280 nm Absorbance Ratios. Biotechniques 18(1):62-63 (1995)
8. Manchester, K. L. Value Of A260/A280 Ratios For Measurement Of The Purity Of Nucleic Acids. Biotechniques 19(2):208-210 (1995)
9. Manchester, K. L. Use Of UV Methods For Measurement Of Protein And Nucleic Acid Concentrations. Biotechniques 20(6):968-970(1996)
10. Wilfinger, W. et al. Effect Of pH and Ionic Strength On The Spectrophotometric Assesment Of Nucleic Acid Purity. Biotechniques. 22(3): 474-481(1997)
11. Teare, J. M. Measurement Of Nucleic Acid Concentrations Using The DYNA Quant And Gene Quant. Biotechniques 22(6): 1170-1174 (1997)
12. Dumar, C. et al. Evaluation Of Quality—Control Criteria For Microarray Gene Expression Analysis. Clinical Chemistry 50(11):1994-2002 (2004)
13. Naderi, A. et al. Expression Microarray Reproducibility Is Improved By Optimizing Purification Steps In RNA Amplification And Labeling. Biomed Central (BMC) Genomics. 5(9): (Jan 30, 2004)
14. Wang, E. RNA Amplification For Successful Gene Profiling Analysis. Journal of Translational Medicine. 3 (28) (2005) doi 10.1186/1479-5876-3-28
15. Ryan, M. et al. Application And Optimization Of Microarray Technologies For Human Postmortem Brain Studies. Biol. Psychiatry 55: 329-336 (2004)
16. Grissom, S. F. et al. A Qualitative Assessment Of Directed -Labeled cDNA Products Prior To Microarray Analysis. BMC Genomics. 6:36 (Mar. 11, 2005) doi 10.1186/1471-2164-6-36
17. Groppe, J. C. and Morse, D. E., Isolation Of Full-Length RNA Templates For Reverse Transcription From Tissues Rich In RNAase And Proteoglycans. Anal. Biochem. 210:337-343 (1993)
18. Chirgwin, J. M. et al. Isolation Of Biologically Active RNA From Sources Enriched In Ribonuclease. Biochem. 18(24):5294-5299 (1979)
19. Glisin, V. et al. RNA Isolated By Cesium Chloride Centifugation. Biochem. 13(12):2633-2637 (1974)
20. Palmer, M. and Prediger, E., Assessing RNA Quality. Ambion Inc. Technote 11(1). Obtained Sep. 26, 2005 at http://www.ambion.com/techlib/tn/111/8.html
21. Is Your RNA Intact? Methods To Check RNA Integrity. Ambion Inc. Technote 8(3). Obtained Sep. 17, 2005 http://www.ambion.com/techlib/tn/83/8313.html
22. Determinants Of RNA Integrity And Purity. Ambion Inc. Technote 11(2) Obtained Sep. 26, 2005 http://www.ambion.com/techlib/tn/112/10.html
23. Choe, S. E. et al. Preferred Analysis Methods For Affymetrix Gene Chips Revealed By A Wholly Defined Control Dataset. Genome Biology 6:R16 (Jan 28, 2005)
24. Byrne, M. C. et al. Preparation Of mRNA For Expression Monitoring. In Current Protocols In Molecular Biol. John Wiley and Sons. 22.2.1-22.2.13 (2000)
25. Bohren, C. F. and Hoffman, D R. In Absorption And Scattering Of Light By Small Particles. Pg 133. John Wiley And Sons (1983)
26. Tinoco, l. et al. Physical Chemistry. Priciples And Applications In Biological Sciences. Third Edition. Pg. 559. Prentice Hall Pub. (1995)
27. Demidov, V. V. and Broude, N. E. DNA Amplification, Current Technologies And Applications. Horizon Bioscience (2004)
28. Kohne, D. and Britten, R. Hydroxyapatite Techniques For Nucleic Acid Reassociation. In Procedures In Nucleic Acid Research Vol 2. ed. Contoni, G. and Davies, D. Harper and Row. Pages 500-512 (1971)

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

While the present invention has been described in terms of a large number of different particular embodiments, it is not intended that the invention be limited to those embodiments. These multiple embodiment descriptions are but a small fraction of the present invention embodiments, and numerous modifications of the described embodiments which are within the scope of the invention will be apparent to those skilled in the art.

For the purpose of explanation, the foregoing explanation used specific nomenclature to assist in understanding of the invention and its many embodiments. However, it will be apparent to one of skill in the art that this nomenclature and specific details are but one way to describe and implement the invention. Thus, the foregoing descriptions of particular embodiments of the present invention are presented for the purpose if illustration and description, and they are not intended to be exhaustive or to limit the invention to the precise forms disclosed. The embodiments presented were selected and described in order to best explain the principles of the present invention and its practical applications, and to thereby enable others skilled in the art to practice the invention and various embodiments with various modifications, as are suited to the particular use contemplated.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values or value range endpoints are provided for embodiments, additional embodiments are described by taking any 2 different values as the endpoints of a range or by taking two different range endpoints from specified ranges as the endpoints of an additional range. Such ranges are also within the scope of the described invention. Further, specification of a numerical range including values greater than one includes specific description of each integer value within that range.

Thus, additional embodiments are within the scope of the invention and within the following claims.

1. (canceled)

2. The method of claim 15 wherein either the nucleic acid prep OD230 or OD280 value is not determined, and either the nucleic acid prep OD260/OD230 ratio or the nucleic acid prep OD260/OD280 ratio value is not determined.

3. The method of claim 15 wherein neither the nucleic acid prep OD230 value nor OD280 value is determined and neither the nucleic acid prep OD260/OD230 ratio value nor OD 260/OD280 ratio value is determined.

4. The method of claim 15, wherein either or both of the nucleic acid prep integrity quality characteristic (IQC) value, and functional homogeneity quality characteristic (FHQC) value, are determined for the nucleic acid prep.

5. The method of claim 15 wherein,
a) The nucleic acid prep is a cell total RNA or isolated mRNA or cell genomic DNA nucleic acid prep, or
b) The nucleic acid prep is a cDNA or cRNA or aRNA or amplified genomic or other DNA nucleic acid prep produced from cell total RNA or isolated mRNA or genomic DNA or other DNA, or
c) The nucleic acid prep is a chemically or enzymatically synthesized RNA or DNA nucleic acid prep.

6. The method of claim 15 wherein a wavelength other than 320 nm is used to detect and quantitate the presence of light scattering substance (LSS) in a nucleic acid solution.

7. The method of claim 15, wherein wavelengths different from but similar to 230 nm, 260 nm, 280 nm, and 320 nm, are used to determine the nucleic acid prep QQC and/or PQC values.

8. The method of claim 15, wherein the purified nucleic acid prep contains a significant amount of one or more low molecular weight contaminant substances.

9. A method for producing improved results for a particular application which uses a nucleic acid prep, comprising
b) using improved nucleic acid prep quality characteristic values for said nucleic acid prep obtained according to the method of claim 15 and said nucleic acid prep in the particular application which utilizes a nucleic acid prep, to produce improved particular application results for said particular application.

10. The method of claim 9 wherein the particular application is a
a) Gene expression analysis assay for determining the number of mRNA or RNA copies per cell for a cell sample, or
b) A gene expression comparison analysis assay for determining the fold change or differential gene expression ratio value for a particular gene in compared cell samples, or
c) A genomic DNA analysis assay for determining the number of particular gene sequences or nucleotide sequence molecules present in a cell sample or other DNA prep, and/or per cell for a cell sample, or
(d) both a) and b).

11. A method for producing improved results for a further particular application which utilize particular application results, comprising
a) utilizing improved particular application results produced according to claim 9 in a further particular application which uses particular application results, thereby produced improved further particular application results.

12. The method of claim 11 wherein the further particular application comprises
a) A data mining method or process, or
b) A systems biology method or process, or
c) both a) and b).

13. (canceled)

14. The method of claim 18 wherein the other particular application comprises one or more of the following applications,
a) A biological application
b) An industrial application
c) An agricultural application
d) A manufacturing application
e) A basic research application
f) A genetic application
g) A product development application
h) A pharmaceutical application such as drug discover, drug evaluation, drug validity, drug toxicity, drug manufacturing, drug description
i) A toxicology evaluation application
j) A medical or veterinary application
k) others

15. A method for producing improved quantitation quality characteristic (QQC) and purity quality characteristic (PQC) results for one or more nucleic acid preps, which are improved in normalization and accuracy and interpretability, relative to prior art produced QQC and PQC results for the one or more nucleic acid preps, comprising,
(a) measuring the nucleic acid prep absorbance values at about 260 nm and at a wavelength characteristic of light scattering (LSWL) which is not absorbed by nucleic acids or proteins, and optionally at about 230 nm or 280 nm or both, for a purified nucleic acid prep which does not contain significant amounts of low molecular weight UV absorbing contaminant molecules in a measuring solution of known composition and pH; and
(b) normalizing said OD230 and OD260 and OD280 absorbance values for the measured absorbance at said wavelength characteristic of light scattering, and
(c) determining a non-light scattering substance-related PQC value for the nucleic acid prep from the normalized OD260/OD230 and OD260/OD280 ratio values, or
(d) converting the nucleic acid prep normalized OD260 value to micrograms of nucleic acid per ml or other concentration units using a conversion factor which is accurate for the nucleic acid prep measuring solution, to produce a nucleic acid prep QQC value, or
(e) both (c) and (d).

16. The method of claim 15, wherein said wavelength characteristic of light scattering is about 320 nm.

17. The method of claim 15, wherein said wavelength characteristic of light scattering is in the range of 305-400 nm.

18. The method of claim 15, wherein said wavelength characteristic of light scattering is equal to or greater than 305 nm.

19. A method for producing improved results for further particular application results, comprising
directly or indirectly utilizing improved particular application results produced according to claim 1 in a further particular application thereby producing improved further particular application results.
